(19) United States
(12) Patent Application Publication
(10) Pub. No.: US 2011/0093009 A1
(43) Pub. Date: Apr. 21, 2011

(54) OTOMY CLOSURE DEVICE

(75) Inventor: William D. Fox, New Richmond, OH (US)

(73) Assignee: Ethicon Endo-Surgery, Inc., Cincinnati, OH (US)

(21) Appl. No.: 12/580,400

(22) Filed: Oct. 16, 2009

(51) Int. Cl. A61B 17/08

(52) U.S. Cl. 606/216

(57) ABSTRACT

A surgical instrument for closing an otomy includes a handle portion, an outer tube, an inner tube, and at least one deployable/retractable tissue engaging hook. The handle portion may comprise an actuator configured to move the inner tube and the at least one deployable/retractable tissue engaging hook. The outer tube may comprise a distal end and a proximal end, and the inner tube may also comprise a distal end and a proximal end. The inner tube may be configured to be movably retained in the outer tube and may be configured to be coupled to the actuator. The deployable/retractable tissue engaging hook may comprise a distal end and a proximal end. The deployable/retractable tissue engaging hook may be configured to be movably retained in the inner tube. The distal end of the deployable/retractable tissue engaging hook may be configured to engage to a portion of tissue.

### BACKGROUND

The present disclosure relates, in general, to surgical devices for closing an otomy in a body lumen and, more particularly, to devices that can be inserted through a natural orifice in the body and used to close an otomy in a gastrointestinal lumen or hollow organ.

Access to the abdominal cavity may be required for diagnostic and therapeutic endeavors for a variety of medical and surgical diseases. Historically, abdominal access has required a laparotomy (open surgery) to provide adequate exposure. Such procedures, which require incisions to be made in the abdomen, are not particularly well-suited for patients that may have extensive abdominal scarring from previous procedures, those persons who are morbidly obese, those individuals with abdominal wall infection, and those patients with diminished abdominal wall integrity, such as patients with burns and skin grafting or a history of internal organ adhesions. Other patients simply do not want to have a scar if it can be avoided.

Minimally invasive procedures are desirable because such procedures can reduce pain and provide relatively quick recovery times as compared with conventional open medical procedures. Many minimally invasive procedures are performed with an endoscope (including without the limitations of laparoscopes). Such procedures permit a physician to position, manipulate, and view medical instruments and accessories inside the patient through a small access opening in the patient's body. Laparoscopy is a term used to describe one such approach using a rigid laparoscope. In this type of procedure, accessory devices are often inserted into a patient through trocars placed through the body wall. The trocar must pass through several layers of overlapping tissue/muscle before reaching the abdominal cavity.

Still less invasive treatments include those that are performed through insertion of an endoscope through a natural body orifice to a treatment region. Examples of procedures which could be done via this approach include, but are not limited to a peritonoscopy, a gastro-jejunostomy, jejunojejunostomy, cholecystectomy, appendectomy, cystoscopy, hysteroscopy, esophagogastroduodenoscopy, and colonoscopy. Many of these procedures employ the use of a flexible endoscope during the procedure. Flexible endoscopes often have a flexible, steerable articulating section near the distal end that can be controlled by the user by utilizing controls at the proximal end. Minimally invasive therapeutic procedures to treat diseased tissue by introducing medical instruments to a tissue treatment region through a natural opening of the patient (e.g., mouth, anus, vagina) are known as Natural Orifice Translumenal Endoscopic Surgery (NOTES)™ procedures. Medical instruments such as endoscopic needles and graspers may be introduced through a channel of a flexible endoscope, which typically has a diameter in the range of approximately 2.5 millimeters (“mm”) (or approximately 0.10 inches (“in”)) to approximately 4.0 mm (or approximately 0.16 in).

Minimally invasive surgical procedures have changed some of the major open surgical procedures such as gastro jejunostomy or jejunojejunostomy, to simple outpatient surgery. Consequently, the patient's recovery time has changed from weeks to days.

Some of the minimally invasive surgical procedures performed may require a surgeon to create an otomy in a gastrointestinal lumen. During NOTES™ or other procedures, where it is necessary to create an opening through the stomach wall or other hollow organs to allow the surgeon to enter a surgical site with an endoscope for a diagnostic or therapeutic procedure, there always comes a time in the procedure when the otomy must be closed using a leak proof method. In the past, devices such as box staplers, band ligators, linear staplers, clips, and T-tags have been used.

Such conventional surgical devices and procedures for closing otomies in body lumen, however, suffer from various shortcomings. Box staplers require multiple firings across tissue edges that are difficult to approximate without counter-traction. This raises concerns in regards to whether the stapled otomy will be leak proof, as there is some technique sensitivity involved. If a box stapler is used following a NOTES procedure, closing the otomy will be a challenging task due to the lack of insufflation within the stomach because the hole in the stomach the surgeon is attempting to close causes air to leak into the peritoneal cavity rather than insufflating the stomach. A hole formed in very thick stomach tissue is difficult to close using a band ligator. The band ligator acts essentially as a rubber band to hold the bunched thick tissue together until healing can occur. Although linear staplers are promising for laparoscopic procedures, they require a 12 mm port to access the otomy, which may be too large for a NOTES™ procedures. In addition, the linear staplers are difficult to position and angulate for an intragastric approach using a NOTES™ procedure. Additionally, linear staplers suffer the same shortcomings as box staplers in that the hole in the stomach prevents it from being inflated and thus cannot create the necessary internal operative space by way of insufflation. While clips may be used to close otomies formed in thinner tissues, clips would be unlikely capable of holding closed the thick stomach walls. T-tags are also problematic in that they present the concern of blind penetrations through the tissue walls which may unintentionally damage other tissue. In addition, the use of current T-tags is time consuming, and the technique is sensitive to use.

Consequently there is a need for an alternative to conventional surgery that eliminates abdominal incisions and incision-related complications by employing an endoscopic technique to treat an abdominal pathology.

There is a further need for a surgical device that can be introduced into the stomach through the mouth and used to close an otomy in a leak proof manner.

The foregoing discussion is intended only to illustrate some of the shortcomings of conventional surgical devices and techniques for closing an otomy in a body lumen using minimally invasive techniques and should not be taken as a disavowal of claim scope.

### FIGURES

The novel features of the various embodiments are set forth with particularity in the appended claims. The various embodiments, however, both as to organization and methods of operation, may be best understood by reference to the following description, taken in conjunction with the accompanying drawings as follows.

FIG. 1 illustrates one embodiment of a surgical instrument inserted through the mouth and esophagus of a patient to close an otomy formed in a wall of the stomach.

FIG. 2 is partial perspective view of one embodiment of a surgical instrument.

FIG. 3 is a cross-sectional view of a distal portion of the surgical instrument of FIG. 2.

FIG. 4 is a side view of a handle portion of the surgical instrument of FIG. 2.

FIG. 5 is a perspective view of the distal portion of FIG. 3 with a plurality of deployable/retractable tissue engaging hooks shown in a retracted position within an inner tube.

FIG. 6 is a perspective view of the distal portion of FIG. 3 with the plurality of deployable/retractable tissue engaging hooks partially extended distally from the inner tube.

FIG. 7 is a perspective view of the distal portion of FIG. 3 with the plurality of deployable/retractable tissue engaging hooks partially extended distally from the inner tube in splayed configuration.

FIG. 8 is a perspective view of the distal portion of FIG. 3 placed adjacent to the otomy with the plurality of deployable/retractable tissue engaging hooks partially extended distally from the inner tube.

FIG. 9 is a perspective view of the distal portion of FIG. 7 with the deployable/retractable tissue engaging hooks engaged with tissue edges of the otomy in which the surgical instrument is retracted proximally through the otomy.

FIG. 10 is a perspective view of the distal portion of FIG. 8 in which the deployable/retractable tissue engaging hooks are engaged with tissue edges of the otomy and in which the surgical instrument is retracted proximally through the otomy.

FIG. 11 is a perspective view of the surgical instrument of FIG. 3 with the plurality of deployable/retractable tissue engaging hooks engaged with a tissue mass formed around the tissue edges retracted proximally into an outer tube.

FIG. 12 is a perspective view of the surgical instrument of FIG. 11 with a suture pulled to cinch the suture around a tissue mass and hermetically secure the tissue mass until healing can occur.

FIG. 13 is a perspective view of the surgical instrument of FIG. 11 in which a proximal end of the suture is removed.

FIG. 14 is a perspective view of the surgical instrument of FIG. 3 with the inner tube extended distally and the plurality of deployable/retractable tissue engaging hooks in the process of being torn from the tissue mass.

FIG. 15 is partial perspective view of one embodiment of a surgical instrument.

FIG. 16 is a perspective view of a distal portion of FIG. 15.

### DESCRIPTION

Before explaining the various embodiments in detail, it should be noted that the embodiments are not limited in their application or use to the details of construction and arrangement of parts illustrated in the accompanying drawings and description. The illustrative embodiments may be implemented or incorporated in other embodiments, variations and modifications, and may be practiced or carried out in various ways. For example, the surgical instrument configurations disclosed below are illustrative only and not meant to limit the scope or application thereof. Furthermore, unless otherwise indicated, the terms and expressions employed herein have been chosen for the purpose of describing the illustrative embodiments for the convenience of the reader and not to limit the scope thereof. The features illustrated or described in connection with one non-limiting embodiment may be combined with the features of other non-limiting embodiments. Such modifications and variations are intended to be included within the scope of the present disclosure.

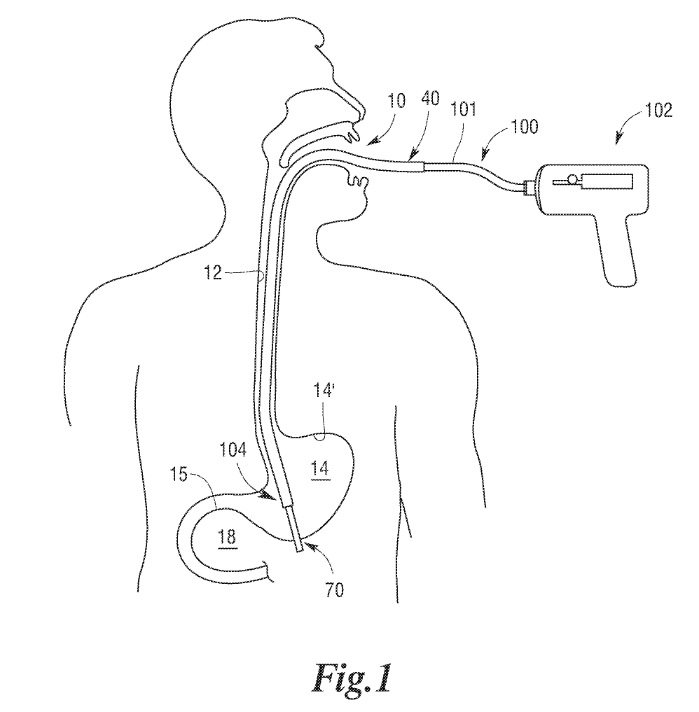

Newer procedures have developed which may even be less invasive than the laparoscopic procedures used in earlier surgical procedures. Many of these procedures employ the use of a flexible endoscope during the procedure. Flexible endoscopes often have a flexible, steerable articulating section near the distal end that can be controlled by the user by utilizing controls at the proximal end. Minimally invasive therapeutic procedures to treat diseased tissue by introducing medical instruments to a tissue treatment region through a natural opening of the patient are known as NOTES™. NOTES™ is a surgical technique whereby operations can be performed trans-orally (as depicted in FIG.1), trans-anally, and/or trans-vaginally.

Certain embodiments will now be described to provide an overall understanding of the principles of the structure, function, manufacture, and use of the devices and methods disclosed herein. One or more examples of these embodiments are illustrated in the accompanying drawings. Those of ordinary skill in the art will understand that the devices and methods specifically described herein and illustrated in the accompanying drawings are non-limiting embodiments and that the scope of the various embodiments is defined solely by the claims. The features illustrated or described in connection with one embodiment may be combined with the features of other embodiments without limitation, and modifications and variations are intended to be included within the scope of the claims.

It will be appreciated that the terms “proximal” and “distal” are used herein with reference to a clinician gripping the surgical instrument. Thus, deployable/retractable tissue engaging hooks are distal with respect to the handle assemblies of the surgical instrument. It will be further appreciated that, for convenience and clarity, spatial terms such as “top” and “bottom” also are used herein with respect to the clinician gripping the handle. However, surgical instruments may be used in many orientations and positions, and these terms are not intended to be limiting and absolute.

FIG. 1 illustrates one embodiment of a surgical instrument 100 inserted through the mouth 10 and esophagus 12 of a patient to close an otomy 70 formed in a wall 14′ of the stomach 14. The surgical instrument 100 may comprise a proximal “handle” portion 102 and a distal portion 104. In one embodiment, the surgical instrument 100 may be used to close an otomy formed in any portion of the body of the patient, such as the esophagus 12, the jejunum 15 and/or any other portion of the patient's gastrointestinal system 18. The otomy 70 may have been formed in a one of a number of body lumens of the patient. The body lumens may comprise any internal body lumens, or portion thereof, which may include the stomach 14, the jejunum 15 or another portion of the small intestine, the large intestine, the esophagus 12, and/or any other body lumen.

The surgical instrument 100 of FIG. 1 may comprise a flexible endoscopic portion 101 which may be inserted into the upper gastrointestinal tract of the patient. The surgical instrument 100 may be configured to flexibly extend through the upper gastrointestinal tract of the patient. The surgical instrument 100 may be flexible to allow the surgical instrument 100 to move along the gastrointestinal tract. In one embodiment, the surgical instrument 100 may be inserted through an over-tube 40 which may have been already inserted into the upper gastrointestinal tract of the patient. FIG. 1 illustrates, in general form, one embodiment of the surgical instrument 100 that can be inserted through a natural orifice such as the mouth 10 and esophagus 12 into the stomach 14 to repair an otomy 70 in the stomach wall. The embodiments, however, are not limited in this context.

In one embodiment, the surgical instrument 100 may be used in conjunction with the over-tube 40. The over-tube 40 may be employed to allow various surgical instruments to be inserted into the body of a patient without each individual surgical instrument rubbing on the esophagus 12 (FIG. 1) of the patient. In use, the over-tube 40 is advanced through the esophagus 12 and acts as a conduit for surgical instruments and protects the esophageal tissue. For example, once the over-tube 40 is placed at a desirable location, individual surgical instruments may be inserted and removed through the central opening of the over-tube 40 without rubbing against the wall of the esophagus 12. The surgical instrument 100 may be inserted into the over-tube 40 with an endoscope (not shown) to allow viewing of the surgical site by the surgeon. Various cameras and/or lighting apparatuses may be inserted into a viewing port of the endoscope to provide the surgeon with a view of the surgical site. In one embodiment, the endoscope may be of a smaller size than the endo scope used in conventional applications as it must fit within the over-tube 40 along with the surgical instrument 100. For example, the over-tube 40 may be formed of a tube with an inner diameter of approximately 14 mm (or approximately 0.55 in) and an outer diameter typically no greater than approximately 22 mm (or approximately 0.9 in), and the surgical instrument 100 may have a diameter of approximately 13 mm (or approximately 0.51 in). In order to fit within the over-tube 40 to provide the viewing of the surgical site, the endo scope typically should have a diameter in the range of approximately 2 mm (or approximately 0.08 in) to approximately 14 mm (or approximately 0.55 in).

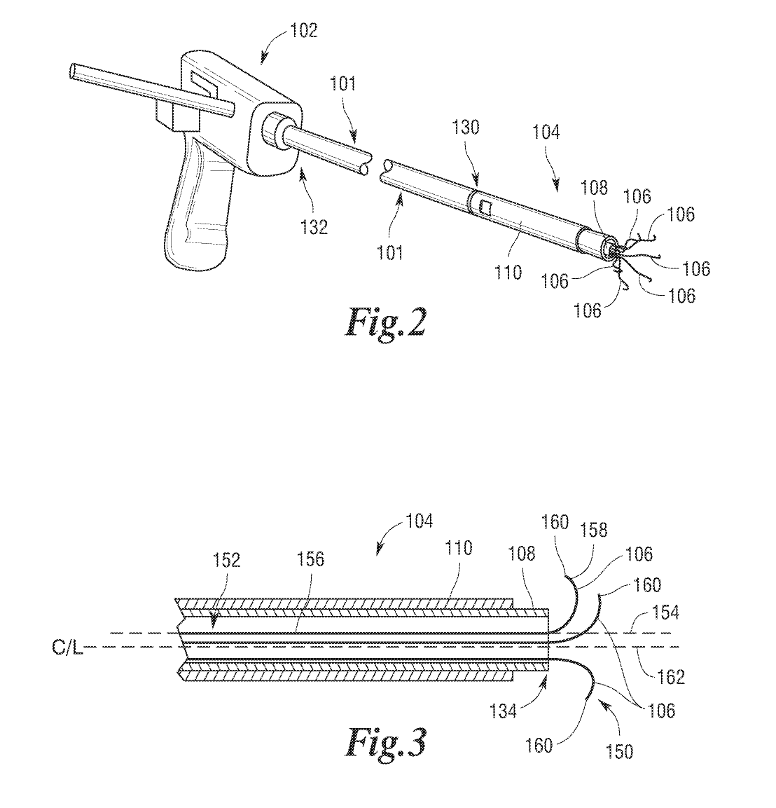

FIG. 2 is partial perspective view of one embodiment of the surgical instrument 100. As previously discussed, in one embodiment, the surgical instrument 100 may comprise a proximal handle portion 102 and a distal portion 104 and further may comprise an outer tube 110. In one embodiment, the surgical instrument 100 may comprise an inner tube 108 defining a longitudinal axis 162 therethrough, and at least one tissue engaging hook 106 at a distal portion 104 of the surgical instrument 100. In one embodiment, the at least one tissue engaging hook 106 may be deployable and retractable, and may be referred to herein as a deployable/retractable tissue engaging hook 106, for example. The outer tube 110 may be hollow. The outer tube 110 may be formed from a flexible material. In various embodiments, the outer tube 110 may be fabricated from, for example, nylon or high density polyethylene plastic. The outer tube 110 may comprise a distal end 130 and a proximal end 132. The distal end 130 and the proximal end 132 may be formed as one piece fabricated from the same material, or may be formed as separate pieces fabricated from the same material or different materials. In various embodiments, the distal end 130 may be fabricated from, for example, medical grade stainless steel or any other suitable material, and the proximal end 132 may be fabricated from, for example, nylon or high density polyethylene plastic. In various embodiments, the distal end 130 may be attached to the proximal end 132 by, for example, suitable adhesive such as cyanoacrylate or epoxy glues, heat seal or light activated adhesives such that a substantially fluid tight seal is established between the distal end 130 and the proximal end 132. The proximal end 132 may be attached to the handle portion 102 by, for example, suitable adhesive such as cyanoacrylate or epoxy glues, heat seal or light activated adhesives.

FIG. 3 is a cross-sectional view of the distal portion 104 of the surgical instrument 100. In one embodiment, the inner tube 108 may be formed from a flexible material. The inner tube 108 may be hollow. In various embodiments, the inner tube 108 may be fabricated from, for example, nylon or high density polyethylene plastic. The inner tube 108 may comprise a distal end 134 and a proximal end (not shown). The distal end 134 and the proximal end may be formed as one piece fabricated from the same material, or may be formed as separate pieces fabricated from the same material or different materials. In various embodiments, the distal end 134 may be fabricated from, for example, medical grade stainless steel or any other suitable material, and the proximal end of the inner tube 108 may be fabricated from, for example, nylon or high density polyethylene plastic. In various embodiments, the distal end 134 may be attached to the proximal end by, for example, suitable adhesive such as cyanoacrylate or epoxy glues, heat seal or light activated adhesives such that a substantially fluid tight seal is established between the distal end 134 and the proximal end. The proximal end of the inner tube 108 may be attached to the handle portion 102 (FIGS. 1, 2) by, for example, suitable adhesive such as cyanoacrylate or epoxy glues, heat seal or light activated adhesives.

In one embodiment, the inner tube 108 may be configured to be movably retained or slidably disposed in the outer tube 110. In one embodiment, the surgical instrument 100 may comprise at least one deployable/retractable tissue engaging hook 106. In various embodiments, the surgical instrument 100 may comprise a plurality of two or more tissue engaging hooks 106 that may be deployable and retractable. For example, in one embodiment the surgical instrument 100 may comprise seven tissue engaging hooks 106.

In one embodiment, the deployable/retractable tissue engaging hooks 106 may comprise a distal end 150 and a proximal end 152. The tissue engaging hooks 106 may be configured to be movably retained or slidably disposed in the inner tube 108. The tissue engaging hooks 106 may be flexible enough to travel along the length of the surgical instrument 100. In one embodiment, the distal end 150 of the tissue engaging hooks 106 may be configured to engage to a portion of tissue. The tissue engaging hooks 106 may comprise a longitudinal extending portion 156 and an arcuate portion 158 at the distal end 150 of the tissue engaging hooks 106. The longitudinal extending portion 156 may comprise a longitudinal axis 154. In one embodiment, the distal end 150 of the tissue engaging hooks 106 may comprise a sharp or tissue penetrating tip 160. For example, the tissue penetrating tip 160 may be formed in a needle shape. The tissue engaging hooks 106 may be fabricated from medical grade stainless steel, nitinol, or polyetheretherketon (PEEK) hypodermic tubing or any other suitable medical grade material which may include metal and/or plastic suitable for medical applications, for example.

In one embodiment, the tissue penetrating tip 160 (FIG. 3) may be chamfered around a periphery of the distal end 150 of the deployable/retractable tissue engaging hook 106. The distal end 150 of the tissue engaging hook 106 may be ground to form the tissue penetrating tip 160. In one embodiment, the tissue penetrating tip 160 may be formed such that the distal end 150 of the tissue engaging hook 106 is cut at an angle. The tissue penetrating tip 160 may be cut and/or ground so that the sharp portion of the tissue penetrating tip 160 is located at the outer edge of the diameter of the distal end 150 of the tissue engaging hook 106. The tissue engaging hook 106 may have an outer diameter in the range of approximately 0.25 mm (or approximately 0.010 in) to approximately 5.0 mm (or approximately 0.20 in).

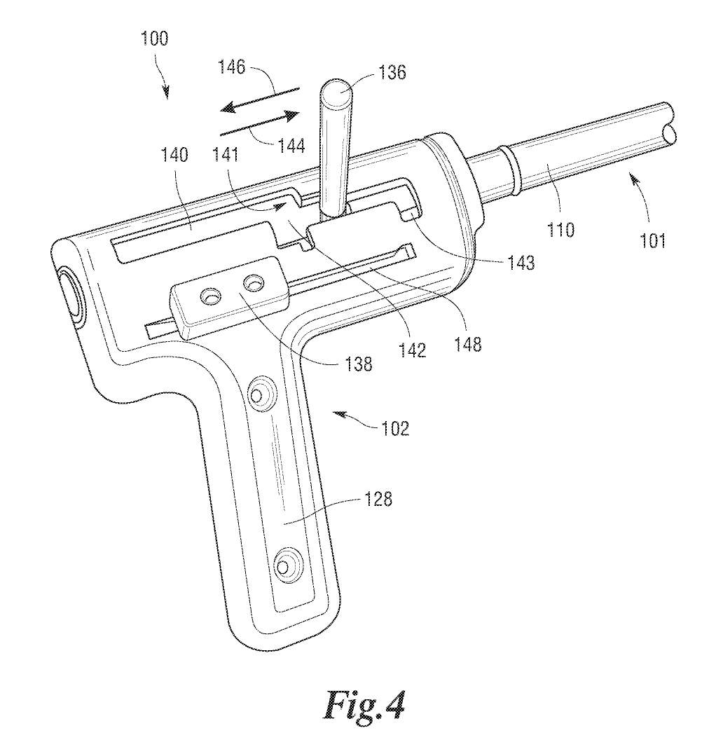

FIG. 4 is a side view of a handle portion 102 of the surgical instrument 100. The handle portion 102 may comprise a grip portion 128, a first actuator 136, and a second actuator 138. The inner tube 108 (FIGS. 2, 3), which is located within the outer tube 110 of the endoscopic portion 101 of the surgical instrument 100, may be configured to be coupled to the first actuator 136. The first actuator 136 may be configured to translate the inner tube 108 and the tissue engaging hooks 106. The first actuator 136 may be configured to translate the inner tube 108 when the first actuator 136 is moved through a first portion 140 of a slot 141 in the handle portion 102. In one embodiment, the slot 141 may comprise the first portion 140 that extends longitudinally along the handle 102, a second portion 142 that extends substantially orthogonally or transversely from the first portion 140, and a third portion 143 that extends longitudinally along the handle 102. The inner tube 108 may be translated distally when the first actuator 136 is translated distally as represented by arrow 144. The inner tube 108 may be translated proximally when the first actuator 136 is translated proximally as represented by arrow 146. In one embodiment, the tissue engaging hooks 106 may be deployed by translating the inner tube 108 with the first actuator 136. In response thereto, the tissue engaging hooks 106 are translated along with the inner tube 108 in the same direction as the inner tube 108, and are thus deployed into the desired location.

The first actuator 136 may be configured to translate, e.g., deploy, the tissue engaging hooks 106 when the first actuator 136 is moved through the third portion 143 of the slot 141 in the handle portion 102 when the first actuators 136 has been moved through the second portion 142. The tissue engaging hooks 106 may be translated distally when the first actuator 136 is translated distally as represented by arrow 144. The tissue engaging hooks 106 may be translated proximally, e.g., retracted, when the first actuator 136 is translated proximally as represented by arrow 146. In one embodiment, the deployable/retractable tissue engaging hooks 106 may be configured to splay, e.g., to spread out, expand, or extend outwardly, from a longitudinal axis 162 (FIG. 3) of the surgical instrument 100 when the tissue engaging hooks 106 are moved distally by the first actuator 136. In one embodiment, the deployable/retractable tissue engaging hooks 106 may be configured to collapse towards the longitudinal axis 162 (FIG. 3) of the surgical instrument 100 when the tissue engaging hooks 106 are moved proximally by the first actuator 136. In various embodiments, a single actuator or a multiple different actuators may be actuated to translate the inner tube 108 and/or the deployable/retractable tissue engaging hooks 106.

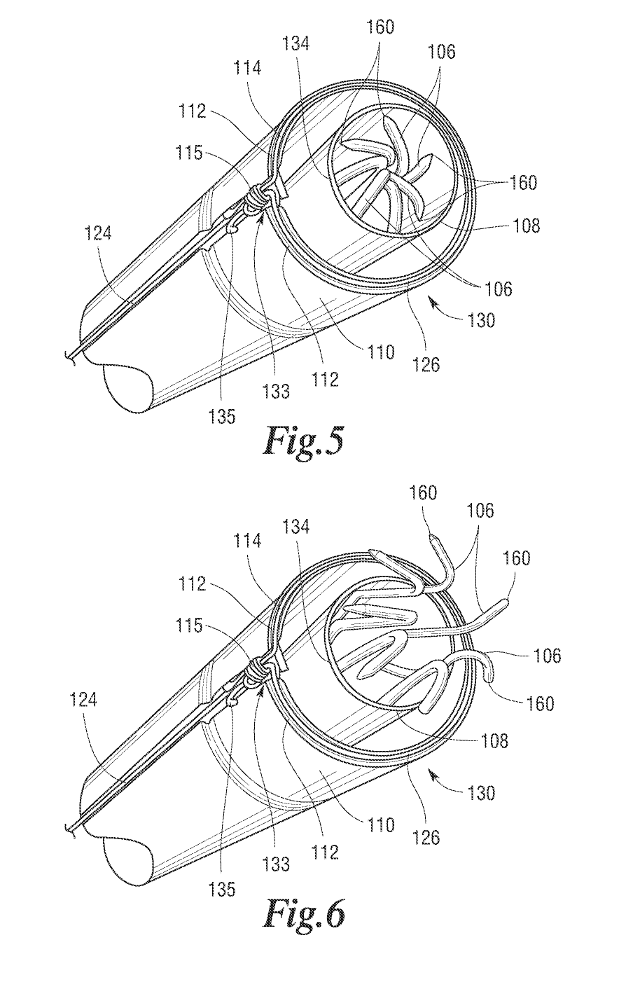

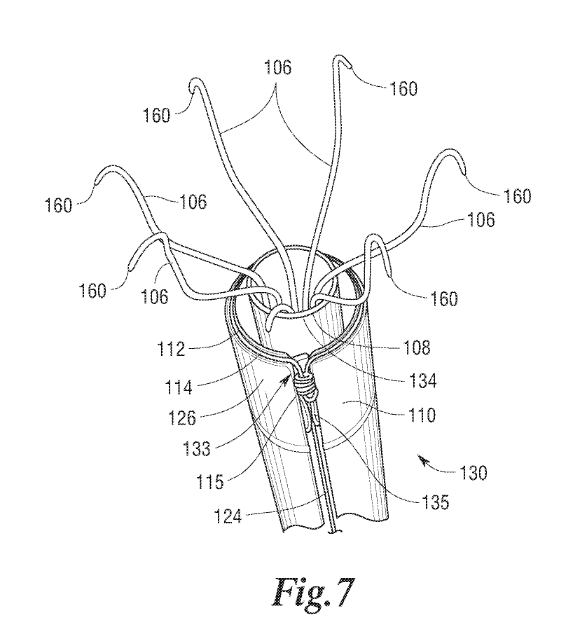

FIG. 5 is a perspective view of the distal portion 104 of the surgical instrument 100 with a plurality of deployable/retractable tissue engaging hooks 106 shown in a retracted position within the inner tube 108. FIG. 6 is a perspective view of the distal portion 104 of the surgical instrument 100 with the plurality of deployable/retractable tissue engaging hooks 106 partially extended distally from the inner tube 108. FIG. 7 is a perspective view of the distal portion 104 of the surgical instrument 100 with the plurality of deployable/retractable tissue engaging hooks 106 partially extended distally from the inner tube 108 in a splayed configuration. In one embodiment, the outer tube 110 may comprise a groove 112 extending around a periphery of the distal end 130 of the outer tube 110. The groove 112 may be configured to receive a suture 114.

With reference briefly to FIGS. 4 and 5, in one embodiment, the suture 114 may be configured to be coupled to the second actuator 138. The second actuator 138 may be configured to translate the suture 114 when the second actuator 138 is moved in a slot 148. The suture 114 may be translated proximally when the second actuator 138 is translated proximally as represented by arrow 146. In one embodiment, the suture 114 may be translated proximally to hermetically secure the tissue until healing can occur. In one embodiment, the suture 114 may be translated distally when the second actuator 138 is translated distally as represented by arrow 144.

With reference now to FIGS. 4-7, in one embodiment, the distal end 130 of the outer tube 110 may comprise an opening 133. The opening 133 may extend proximally towards the handle portion 102. The opening 133 may be configured to receive a knot 115 and a proximal end 124 of the suture 114. The opening 133 may extend along the length of the outer tube 110 to the handle portion 102 and may be configured to allow the proximal end 124 of the suture 114 to extend along the length of the outer tube 110 to the handle portion 102. The configuration of the opening 133 may allow a surgeon to translate the proximal end 124 of the suture 114 proximally when the surgeon desires to tighten a distal end 126 of the suture 114, which may be configured as a noose, e.g., a loop with a running knot. For example, the surgeon may translate the proximal end 124 of the suture when the surgeon desires to cinch, snare, or secure a portion of tissue with the distal end 126 of the suture 114 as to form a hermetic seal. The proximal end 124 of the suture 114 may be translated by the second actuator 138 or by hand (not shown).

In one embodiment, a suture cut-off device 135 may be configured to be coupled to the second actuator 138. The second actuator 138 may be configured to translate the suture cut-off device 135 when the second actuator 138 is moved in a slot 148. The suture cut-off device 135 may be translated distally when the second actuator 138 is translated distally as represented by arrow 144. The suture cut-off device 135 may be translated proximally when the second actuator 138 is translated proximally as represented by arrow 146. The suture cut-off device may be fabricated from medical grade stainless steel or any other suitable medical grade material which may include metal and/or plastic suitable for medical applications, for example.

In one embodiment, the suture cut-off device 135 may be located within the opening 133. The suture cut-off device 135 may be fixed in a stationary position in the opening 133. In one embodiment, the suture cut-off device 135 may be configured to translate within the opening, for example, through the use of the second actuator 138 and/or any other suitable actuator. In one embodiment, the suture cut-off device 135 may be configured to remove the proximal end 124 of the suture 114 from a distal end 126 of the suture 114. The suture cut-off device 135 may comprise a knife or any other suitable device for separating the two portions of the suture 114, for example.

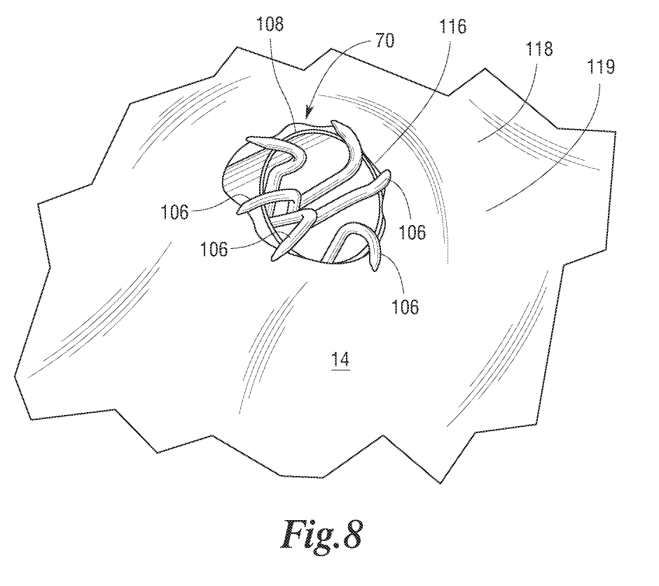

FIG. 8 is a perspective view of the distal portion 104 of the surgical instrument 100 placed adjacent to the otomy 70 with the plurality of the tissue engaging hooks 106 partially extended distally from the inner tube 108. A method of using the surgical instrument 100 to close an otomy is illustrated in FIGS. 8-14. As previously discussed, the surgical instrument 100 may be inserted trans-orally through the esophagus 12 (FIG. 1) to reach an otomy 70 in the patient, for example, in the stomach 14. The surgical instrument 100 may extend into the stomach 14 of the patient. As shown in FIG. 1, the surgical instrument 100 may be extended until it contacts a portion of the stomach 14 wall 14′. The distal portion 104 of the surgical instrument 100 may be translated through the gastrointestinal tract of the patient until the distal portion 104 is placed adjacent a proximal side of the otomy 70. The otomy 70 may be defined by tissue edges 116 formed in the tissue 118. In one embodiment, the tissue engaging hooks 106 may be extended distally through the otomy 70 to the distal side 119 of the otomy 70. In one embodiment, extending the tissue engaging hooks 106 may comprise actuating the first actuator 136 (FIG. 4) to extend the tissue engaging hooks 106. In one embodiment, the tissue engaging hooks 106 may splay, or extend both along the longitudinal axis 162 and away or outwardly from the longitudinal axis 162 of the surgical instrument 100, on the distal side 119 of the otomy 70. In various other embodiments, the tissue engaging hooks 106 may extend along the longitudinal axis 162 of the surgical instrument without splaying.

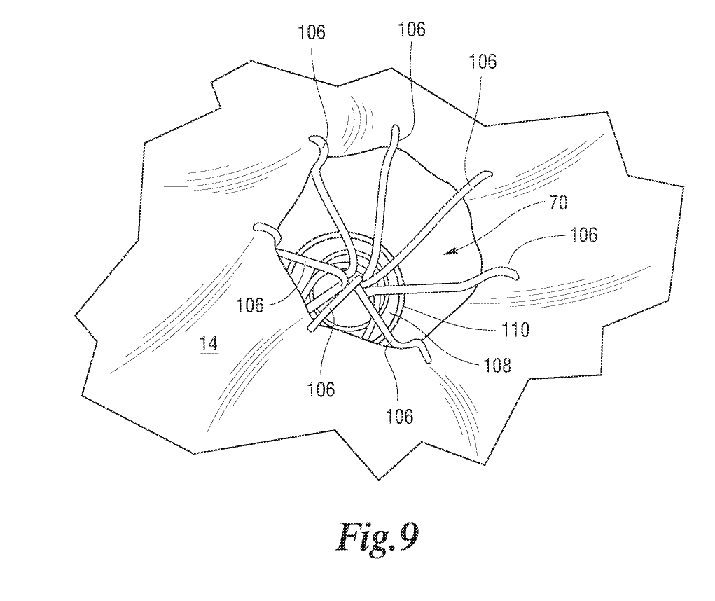

FIG. 9 is a perspective view of the distal portion 104 of the surgical instrument 100 with the tissue engaging hooks 106 engaged with tissue edges 116 of the otomy 70 in which the surgical instrument 100 is retracted proximally through the otomy 70. In one embodiment, the tissue engaging hooks 106 may engage the tissue edges 116. In one embodiment, the tissue penetrating tips 160 may puncture the tissue 118 proximal to the tissue edges 116. In one embodiment, each tissue penetrating tips 160 may puncture the tissue 118 proximal to the tissue edges 116. In various embodiments, only a portion of the total number of tissue penetrating tips 160 may puncture the tissue 118 proximal to the tissue edges 116. The tissue engaging hooks 106 may be translated proximally from the distal side 119 of the otomy 70 until the tissue penetrating tips 160 of the tissue engaging hooks 106 puncture the tissue edges 116.

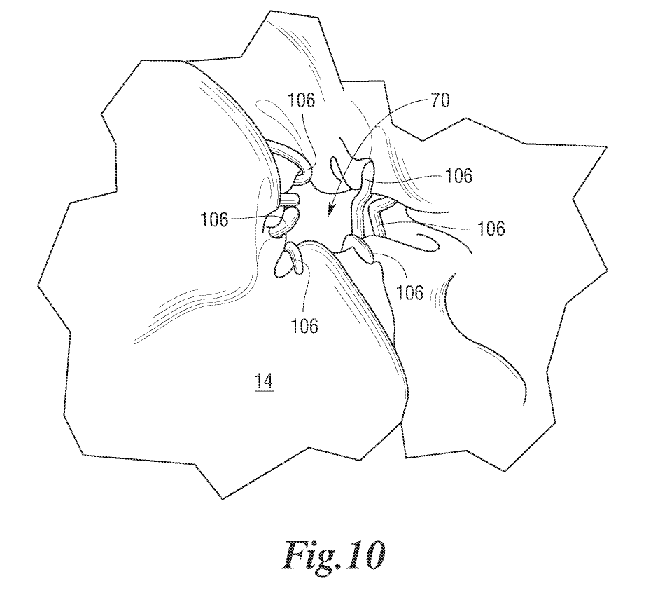

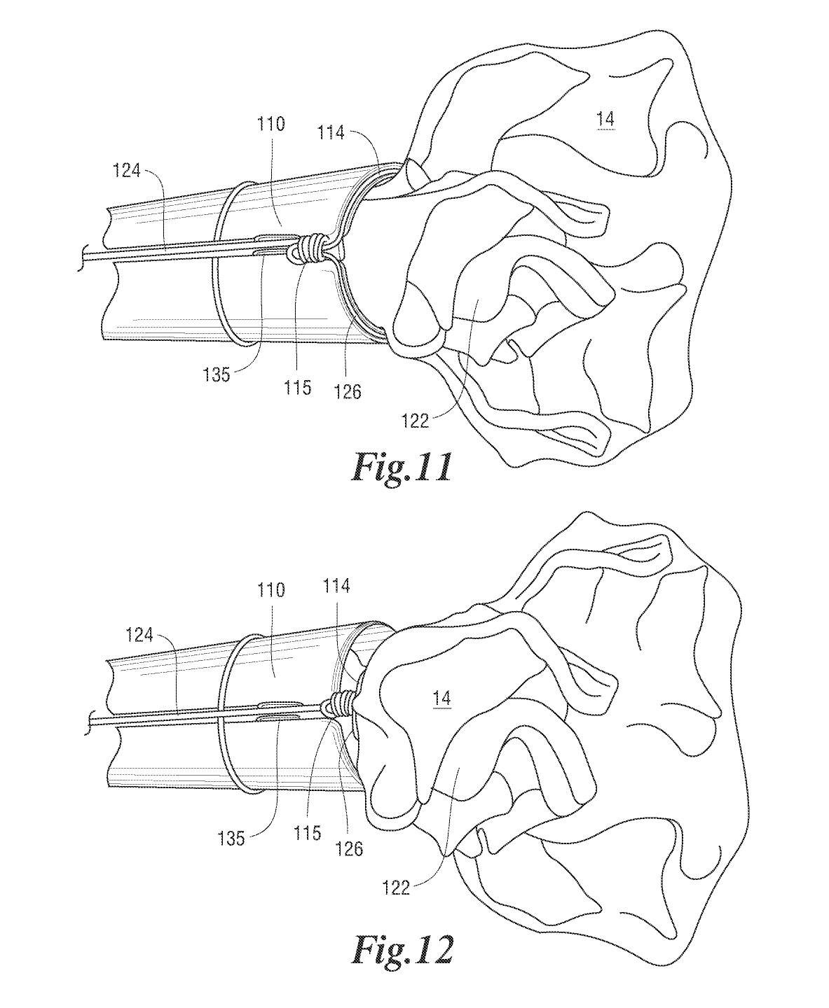

FIG. 10 is a perspective view of the distal portion 104 of the surgical instrument 100 in which the tissue engaging hooks 106 are engaged with tissue edges 116 of the otomy 70 and in which the surgical instrument 100 is retracted proximally through the otomy 70. As shown, the plurality of tissue engaging hooks 106 are retracted proximally to bunch the tissue edges 116 towards the longitudinal axis 162 of the surgical instrument 100. The tissue engaging hooks 106 may be retracted to bunch the tissue 118 surrounding the tissue edges 116 towards the longitudinal axis 162 of the surgical instrument 100 forming a tissue mass 122 (FIG. 11). In one embodiment, retracting the tissue engaging hooks 106 may comprise actuating the first actuator 136 (FIG. 4) to retract the deployable/retractable tissue engaging hooks 106. The deployable/retractable tissue engaging hooks 106 may retract in an opposite manner to the manner in which they were extended. In one embodiment, the deployable/retractable tissue engaging hooks 106 may collapse, both along the longitudinal axis 162 and towards the longitudinal axis 162 of the surgical instrument 100. In various other embodiments, the deployable/retractable tissue engaging hooks 106 may retract along the longitudinal axis 162 of the surgical instrument without collapsing towards the longitudinal axis 162 of the surgical instrument 100.

FIG. 11 is a perspective view of the distal portion 104 with the plurality of deployable/retractable tissue engaging hooks 106 engaged with a tissue mass 122 formed around the tissue edges 116 retracted proximally into the outer tube 110. The bunching of the tissue edges 116 may form the tissue mass 122 on the proximal side of the otomy 70. In one embodiment, when the tissue engaging hooks 106 are retracted near the distal end 134 of the inner tube 108, the inner tube 108 may be retracted by further actuating the first actuator 136 proximally, as previously discussed. The first actuator 136 may be actuated to retract the inner tube 108 and thus retract the tissue edges 116. The inner tube 108 and the tissue engaging hooks 106 may be retracted substantially simultaneously through the actuation of the first actuator 136 or may be retracted separately. The inner tube 108 and the tissue engaging hooks 106 may be retracted to engage the tissue mass 122 in the outer tube 110.

FIG. 12 is a perspective view of the distal portion 104 of the surgical instrument 100 with the suture 114 pulled proximally to cinch the suture 114 around the tissue mass 122 and hermetically secure the tissue mass 122 until healing can occur. In one embodiment, the proximal end 126 of the suture 114 may be pulled proximally to cinch the suture 114 around the tissue mass 122, as discussed earlier. This may allow the tissue mass 122 to be hermetically secured until healing can occur.

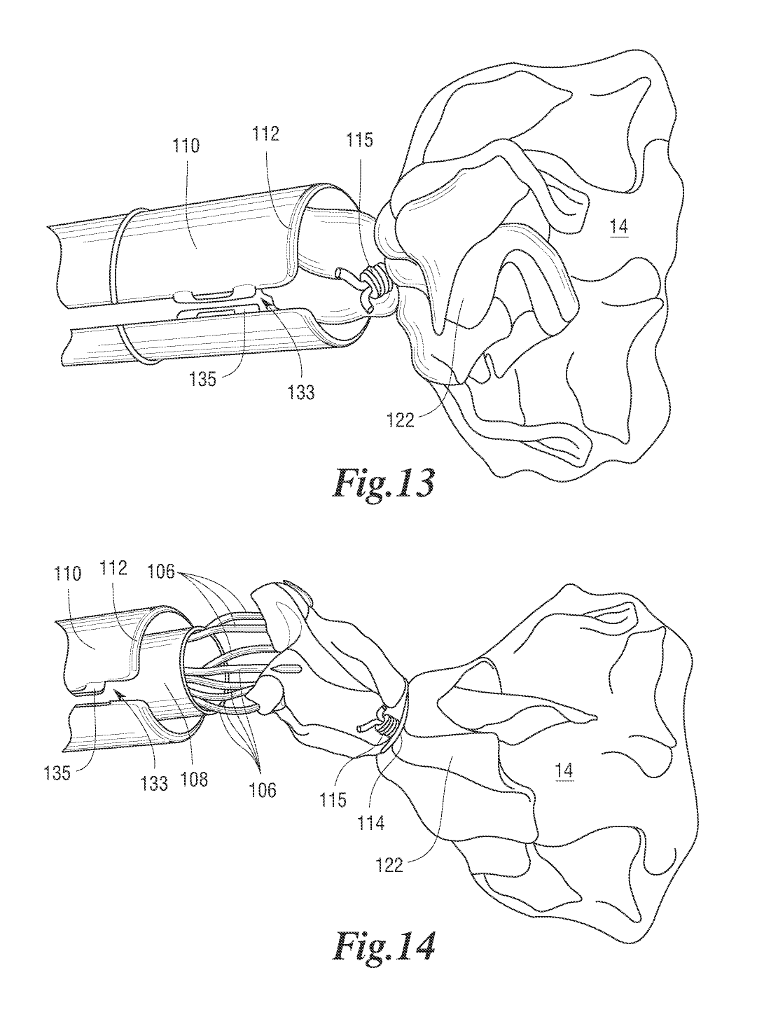

FIG. 13 is a perspective view of the distal portion 104 of the surgical instrument 100 in which the proximal end 124 of the suture 114 is removed. In one embodiment, the proximal end 124 of the suture 114 may be removed by the surgeon. In one embodiment, the proximal end 124 of the suture 114 may be removed by cutting the proximal end 124 of the suture 114. For example, the proximal end 124 of the suture 114 may be removed using the suture cut-off device 135. In one embodiment, once the proximal end 124 of the suture 114 has been removed, the surgical instrument 100 may be retracted from proximal side of the otomy 70.

FIG. 14 is a perspective view of the distal portion 104 of the surgical instrument 100 with the inner tube 108 extended distally and the plurality of tissue engaging hooks 106 in the process of being torn from the tissue mass 122. In one embodiment, the surgical instrument 100 may be retracted from the proximal side of the otomy 70 by tearing the tissue engaging hooks 106 from the tissue edges 116. As shown in FIG. 14, the inner tube 108 and the deployable/retractable tissue engaging hooks 106 may be extended distally, in the manner previously discussed, prior to tearing the tissue engaging hooks 106 from the tissue mass 122. In various embodiments, the tissue engaging hooks 106 may be torn from the tissue mass 122 without extending the tissue engaging hooks 106 and/or the inner tube 108. In one embodiment, the tissue engaging hooks 106 may include a sharpened edge which may allow the tissue engaging hooks 106 to cut through the tissue edges 116. Once the tissue engaging hooks 106 have been torn from the tissue mass 122, the surgical instrument 100 may be retracted proximally from gastrointestinal tract and proximally from the mouth 10 of the patient.

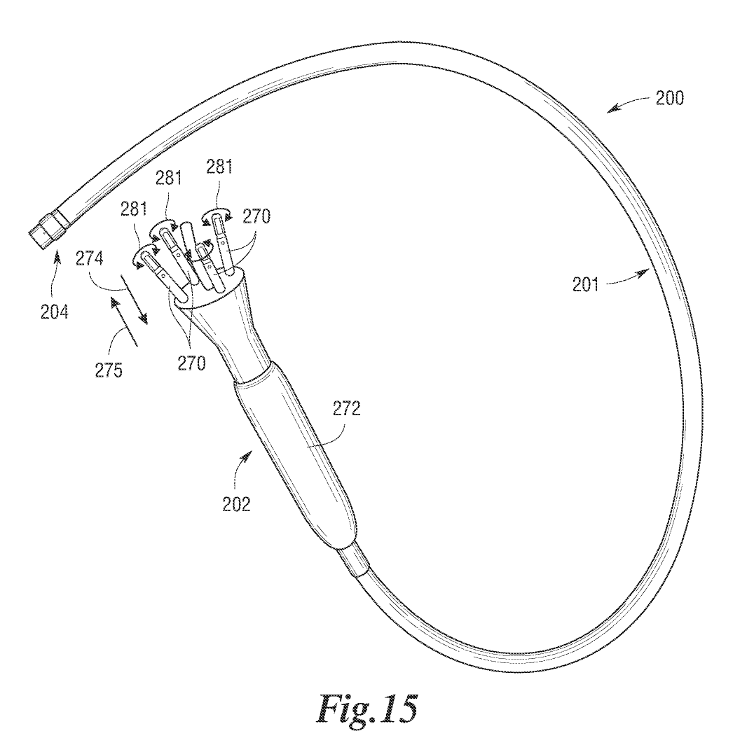

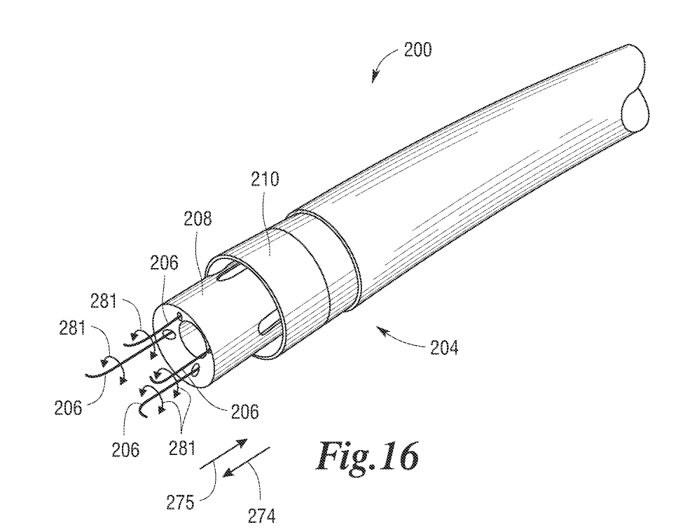

FIG. 15 is partial perspective view of one embodiment of a surgical instrument 200. FIG. 16 is a perspective view of a distal portion 204 of the surgical instrument 200. The components with corresponding reference numerals (e.g., 108, 208) can have the same or a similar structure and function as previously discussed, unless otherwise noted. As such, for the sake of brevity these components will not be discussed in detail again here. The surgical instrument 200 may comprise a distal portion 204, a handle portion 202, an outer tube 210, an inner tube 208, and at least one tissue engaging hook 206 that may be deployed and/or retracted. In one embodiment, each of the tissue engaging hooks 206 may be configured to rotate about a longitudinal axis 254 of the tissue engaging hooks 206. Each of the tissue engaging hooks 206 may be configured to be individually translated distally. In one embodiment, each of the retractable tissue engaging hooks 206 may be configured to be attached to an actuator 270.

In one embodiment, as depicted in FIGS. 15 and 16, the surgical instrument 200 may comprise four actuators 270 and four deployable/retractable tissue engaging hooks 206, for example. In various embodiments, the surgical instrument 200 may comprise any number of actuators 270 and any number of corresponding tissue engaging hooks 206. The actuators 270 may be configured to translate the tissue engaging hooks 206 when the actuators 270 are translated distally, as indicated by arrow 274. In one embodiment, the actuators 270 may be spring-loaded to be automatically translated proximally, in the direction indicated by arrow 275, when the actuator 270 is released by an operator. In one embodiment, the actuators 270 may be translated proximally by the operator pulling a handle (not shown) proximally.

In one embodiment, referring to FIGS. 15 and 16, each of the tissue engaging hooks 206 may be rotated about a longitudinal axis 262 of the tissue engaging hooks 206 in the direction indicated by arrow 281 when each of the actuators 270 are rotated about a longitudinal axis of the actuators 270. In one embodiment, the surgical instrument 200 may comprise a tube actuator 272. The tube actuator 272 may be configured to translate the inner tube 208. In one embodiment, the tube actuator 272 may be configured to translate the inner tube 208 and deployable/retractable tissue engaging hooks 206 substantially simultaneously.

The device which is intended to be protected is not to be construed as limited to the particular embodiments disclosed. The embodiments are therefore to be regarded as illustrative rather than restrictive. Variations and changes may be made by others without departing from the scope of the described embodiments. Accordingly, it is expressly intended that all such equivalents, variations and changes which fall within the scope of the described embodiments as defined in the claims be embraced thereby.

The devices disclosed herein can be designed to be disposed of after a single use, or they can be designed to be used multiple times. In either case, however, the device can be reconditioned for reuse after at least one use. Reconditioning can include any combination of the steps of disassembly of the device, followed by cleaning or replacement of particular pieces, and subsequent reassembly. In particular, the device can be disassembled, and any number of the particular pieces or parts of the device can be selectively replaced or removed in any combination. Upon cleaning and/or replacement of particular parts, the device can be reassembled for subsequent use either at a reconditioning facility, or by a surgical team immediately prior to a surgical procedure. Those skilled in the art will appreciate that reconditioning of a device can utilize a variety of techniques for disassembly, cleaning/replacement, and reassembly. Use of such techniques, and the resulting reconditioned device, are all within the scope of the present disclosure.

Preferably, the various embodiments described herein will be processed before surgery. First, a new or used instrument is obtained and if necessary cleaned. The instrument can then be sterilized. In one sterilization technique, the instrument is placed in a closed and sealed container, such as a plastic or TYVEK® bag. The container and instrument are then placed in a field of radiation that can penetrate the container, such as gamma radiation, x-rays, or high-energy electrons. The radiation kills bacteria on the instrument and in the container. The sterilized instrument can then be stored in the sterile container. The sealed container keeps the instrument sterile until it is opened in the medical facility.

Although various embodiments have been described herein, many modifications and variations to those embodiments may be implemented. For example, different types of deployable/retractable tissue engaging hooks may be employed. In addition, combinations of the described embodiments may be used. Also, where materials are disclosed for certain components, other materials may be used. The foregoing description and following claims are intended to cover all such modification and variations. It should be apparent, however, that various modifications, alterations and adaptations to those embodiments may occur to persons skilled in the art. For example, a single component may be replaced by multiple components, and multiple components may be replaced by a single component, to perform a given function or functions. This disclosure is therefore intended to cover all such modifications, alterations and adaptations without departing from the scope of the appended claims.

While the present disclosure illustrates and describes several embodiments in considerable detail, it is not the intention of the applicant to restrict or in any way limit the scope of the appended claims to such detail. Additional advantages and modifications may readily appear to those skilled in the art. Those of ordinary skill in the art will readily appreciate the different advantages provided by these various embodiments. While the various surgical instruments have been herein described in connection with the closing of an otomy through a patient's mouth, those of ordinary skill in the art will readily appreciate that the unique and novel features of the various embodiments may be effectively employed in connection with closing an otomy which may be accessed through other natural orifices in the patient. In addition, it is conceivable that the various embodiments could have utility in some laparoscopic surgical procedures and therapies.

Any patent, publication, or other disclosure material, in whole or in part, that is said to be incorporated by reference herein is incorporated herein only to the extent that the incorporated materials does not conflict with existing definitions, statements, or other disclosure material set forth in this disclosure. As such, and to the extent necessary, the disclosure as explicitly set forth herein supersedes any conflicting material incorporated herein by reference. Any material, or portion thereof, that is said to be incorporated by reference herein, but which conflicts with existing definitions, statements, or other disclosure material set forth herein will only be incorporated to the extent that no conflict arises between that incorporated material and the existing disclosure material.

What is claimed is:

1. A surgical apparatus comprising:
an outer flexible tube comprising a distal end and a proximal end;
an inner flexible tube comprising a distal end and a proximal end, the inner flexible tube is configured to be movably retained in the outer flexible tube, the inner flexible tube is configured to be coupled to a first actuator configured to move the inner flexible tube; and
at least one tissue engaging hook comprising a distal end and a proximal end, the tissue engaging hook is configured to be movably retained in the inner flexible tube, the distal end of the at least one tissue engaging hook is configured to engage a portion of tissue, and the at least one tissue engaging hook is configured to be coupled to a second actuator configured to move the at least one tissue engaging hook.

2. The surgical apparatus of claim 1, wherein the at least one tissue engaging hook comprises a plurality of tissue engaging hooks.

3. The surgical apparatus of claim 2, wherein each of the plurality of tissue engaging hooks are configured to rotate about a longitudinal axis of the tissue engaging hook and are each configured to be individually translated.

4. The surgical apparatus of claim 1, wherein the at least one tissue engaging hook comprises a longitudinal extending portion and an arcuate portion at the distal end of the at least one tissue engaging hook.

5. The surgical apparatus of claim 1, wherein the distal end of the at least one tissue engaging hook comprises a tissue penetrating tip.

6. The surgical apparatus of claim 1, wherein the at least one tissue engaging hook is configured to splay from a longitudinal axis of the surgical apparatus when the at least one tissue engaging hook is moved distally by the second actuator.

7. The surgical apparatus of claim 1, wherein the at least one tissue engaging hook is configured to collapse towards the longitudinal axis of the surgical apparatus when the at least one tissue engaging hook is moved proximally by the second actuator.

8. The surgical apparatus of claim 1, wherein the outer flexible tube comprises a groove extending around a periphery of the distal end of the outer flexible member, the groove configured to receive a suture.

9. The surgical apparatus of claim 8, wherein the outer flexible tube comprises an opening at the distal end of the outer flexible tube extending proximally, and wherein the opening is configured to receive a knot of the suture.

10. The surgical apparatus of claim 9, wherein the opening comprises a suture cut-off device.

11. A surgical instrument for closing an otomy, the surgical instrument comprising:
a handle portion, the handle portion comprising a first actuator and a second actuator;
an outer flexible tube comprising a distal end and a proximal end;
an inner flexible tube comprising a distal end and a proximal end, the inner flexible tube is configured to be movably retained in the outer flexible tube, the inner flexible tube configured to be coupled to the first actuator, the first actuator configured to move the inner flexible tube; and
at least one tissue engaging hook comprising a distal end and a proximal end, the tissue engaging hook is configured to be movably retained in the inner flexible tube, the distal end of the at least one tissue engaging hook is configured to engage to a portion of tissue, and the at least one tissue engaging hook is configured to be coupled to the second actuator, the second actuator configured to move the at least one tissue engaging hook.

12. The surgical instrument of claim 11, wherein the at least one tissue engaging hook comprises a plurality of tissue engaging hooks.

13. The surgical instrument of claim 11, wherein the at least one tissue engaging hook comprises a longitudinal extending portion and an arcuate portion at the distal end of the at least one tissue engaging hook.

14. The surgical instrument of claim 13, wherein the distal end of the at least one tissue engaging hook comprises a tissue penetrating tip.

15. The surgical instrument of claim 11, wherein the at least one tissue engaging hook is configured to splay from a longitudinal axis of the surgical apparatus when the at least one tissue engaging hook is moved distally by the second actuator.

16. The surgical instrument of claim 11, wherein the at least one tissue engaging hook is configured to collapse towards the longitudinal axis of the surgical apparatus when the at least one tissue engaging hook is moved proximally by the second actuator.

17. The surgical instrument of claim 11, wherein the outer flexible tube comprises a groove extending around a periphery of the distal end of the outer flexible member, the groove configured to receive a suture.

18. The surgical instrument of claim 17, wherein the outer flexible tube comprises an opening at the distal end of the outer flexible tube extending proximally comprising a suture cut-off device, the opening configured to receive a knot of the suture.

19. A method of closing an otomy, the method comprising:
placing a distal end of a surgical instrument adjacent a proximal side of the otomy, the otomy defined by tissue edges;
extending distally a plurality of tissue engaging hooks through the otomy such that the tissue engaging hooks splay on a distal side of the otomy;
engaging the tissue edges with the tissue engaging hooks;
retracting the tissue engaging hooks to bunch tissue surrounding the tissue edges towards a longitudinal axis of the surgical instrument forming a tissue mass;
retracting an inner tube to engage tissue in an outer tube;
pulling a proximal end of a suture to cinch the suture around the tissue mass to hermetically secure the tissue mass until healing can occur;
removing the proximal end of the suture; and
retracting the surgical device from proximal side of the otomy.

20. The method of claim 19,
wherein extending the plurality of tissue engaging hooks comprises actuating a first actuator to extend the plurality of tissue engaging hooks;
wherein engaging the tissue edges comprises translating the plurality of tissue engaging hooks proximally until the plurality of tissue engaging hooks puncture the tissue edges;
wherein retracting the inner tube comprises actuating the first actuator to retract the tissue edges;
wherein removing the proximal end of the suture comprises cutting the proximal end of the suture; and
wherein retracting the surgical device from the proximal side of the otomy comprises tearing the plurality of tissue engaging hooks from the tissue edges.
